(19) United States
(12) Patent Application Publication
(10) Pub. No.: US 2007/0112402 A1
(43) Pub. Date: May 17, 2007

(54) Electrode systems and related methods for providing therapeutic differential tissue stimulation

(75) Inventors: Warren M. Grill, Chapel Hill, NC (US); Xuefeng F. Wei, Durham, NC (US)

Correspondence Address: JENKINS, WILSON, TAYLOR & HUNT, P. A., 3100 TOWER BLVD, SUITE 1200, DURHAM, NC, 27707, US

(73) Assignee: Duke University

(21) Appl. No.: 11/583,379

(22) Filed: Oct. 19, 2006

Related U.S. Application Data
(60) Provisional application No. 60/728,359, filed on Oct. 19, 2005.

(51) Int. Cl. A61N 1/00

(52) U.S. Cl. 607/115

(57) ABSTRACT

Electrode Systems and Related Methods for Providing Therapeutic Differential Tissue Stimulation. According to one aspect, an electrode system for providing therapeutic differential tissue stimulation includes an electrode body. A plurality of electrode segments are positioned along the electrode body. The electrode segments are predetermined shapes and sizes that produce a predetermined electrical field for stimulating predetermined portions of tissue, and each of the electrode segments includes an outer surface which is exposed from the electrode body for coupling to tissue. A lead is connected to each of the electrode segments for use in applying a common electrical signal to the electrode segments.

### RELATED APPLICATIONS

This non-provisional patent application claims the benefit of U.S. Provisional Application No. 60/728,359, filed Oct. 19, 2005, the disclosure of which is incorporated by reference herein in its entirety.

### GRANT STATEMENT

The presently disclosed subject matter was made with U.S. Government support under Grant No. R01-NS-40894 awarded by the National Institutes of Health. Thus, the U.S. Government has certain rights in the presently disclosed subject matter.

### TECHNICAL FIELD

The subject matter described herein relates to electrode stimulation. More particularly, the subject matter described herein relates to electrode systems and methods for providing therapeutic differential tissue stimulation.

### BACKGROUND

Electrical stimulation of the nervous system is a technique used to restore function to individuals with neurological impairment. For example, deep brain stimulation (DBS) uses high frequency electrical stimulation of the thalamus or basal ganglia (i.e., subthalamic nucleus (STN), internal segment of the globus pallidus) to treat movement disorders, and has rapidly emerged as an alternative to surgical lesions. Although the mechanisms of the action of DBS are still unclear, the efficacy of DBS therapy requires localizing the current delivery to specific populations of neurons. Similarly, spinal cord stimulation (SCS) uses electrical stimulation of the dorsal roots and/or the dorsal columns of the spinal cord to treat pain and angina. Again, although the precise mechanisms of action of SCS are unknown, the efficacy of SCS requires localizing stimulation to specific populations of neurons.

The design of electrodes is important for the controlled activation of populations of neurons. In particular, electrode geometry can affect the spatial distribution of current density over the electrode surface, a cofactor with charge in stimulation induced neural damage. Further, electrode geometry can affect the pattern of neural excitation by determining the electric field generated in tissue medium surrounding the electrode. The electrode design can also affect electrode impedance, which impacts power consumption. These elements are linked in that current density (J) and electric field (E) are related by Ohm's law, which is set forth in the following equation:
J=σ·E
The impedance (Z) depends on the current density distribution over the electrode surface, as set forth in the following equation:

$$Z = \frac{V}{{\int }_{S} J \cdot ⅆ S} ,$$

wherein V is the potential drop across the electrode-electrolyte interface and S is the electrode surface area.

The applied stimulation of electrical fields by using electrodes in the nervous system can produce desired clinical effects. However, such stimulation can also produce unwanted side effects. In many cases, the ability to produce optimal clinical effects is limited by production of side effects. Therefore, it is desirable to provide beneficial electrical stimulation of the nervous system with reduced or negligible side effects.

While significant effort has been put into finding optimal anatomical targets for DBS, there has been limited development in the design of DBS electrodes. It would be beneficial to have electrodes that allow better control of the distribution of the electrical field surrounding the electrodes. DBS therapy may be improved by providing electrodes that provide better control of the electrical field distribution.

Accordingly, there exists a need for electrode systems and methods that provide improved stimulation of target tissue with reduced or negligible side effects.

### SUMMARY

According to one aspect, the subject matter described herein includes electrode systems and methods for providing therapeutic differential tissue stimulation. According to one aspect, an electrode system for providing therapeutic differential tissue stimulation includes an electrode body. A plurality of electrode segments are positioned along the electrode body. The electrode segments are predetermined shapes and sizes that produce a predetermined electrical field for stimulating predetermined portions of tissue, and each of the electrode segments includes an outer surface which is exposed from the electrode body for coupling to tissue. A lead is connected to each of the electrode segments for use in applying a common electrical signal to the electrode segments.

The subject matter described herein may be implemented using a computer program product comprising computer executable instructions embodied in a computer-readable medium. Exemplary computer-readable media suitable for implementing the subject matter described herein include chip memory devices, disk memory devices, programmable logic devices, application specific integrated circuits, and downloadable electrical signals. In addition, a computer-readable medium that implements the subject matter described herein may be located on a single device or computing platform or may be distributed across multiple devices or computing platforms.

### BRIEF DESCRIPTION OF THE DRAWINGS

Preferred embodiments of the subject matter described herein will now be explained with reference to the accompanying drawings of which:

FIG. 1 is a block diagram of an electrode system for providing therapeutic differential tissue stimulation according to an embodiment of the subject matter described herein;

FIG. 2A is a perspective view of an electrode body of the electrode system shown in FIG. 1;

FIG. 2B is a cross-sectional view through an insulating section of the electrode body shown in FIG. 2A;

FIG. 2C is a cross-sectional view through an electrode segment of the electrode body shown in FIG. 2A;

FIG. 3A is a perspective view of a portion of an electrode body including an electrode segment having non-planar ends according to an embodiment of the subject matter described herein;

FIG. 3B is a top view of a portion of the electrode body shown in FIG. 3A;

FIGS. 4A-4E are top plan views of portions of electrode bodies including one or more electrode segments having predetermined shapes and sizes according to the subject matter described herein;

FIG. 5 is a three dimension (3D) numerical computer model of the geometry of a 4-segment electrode system model according to an embodiment of the subject matter described herein;

FIG. 6 is a 3D graph of computer simulation results illustrating the distribution of potentials generated in a tissue medium by the segmented electrode shown in FIG. 4C;

FIGS. 7A and 7B are graphs of computer simulation results of the second spatial differences of the potentials, Δ²Vₑ/Δx² and Δ²Vₑ/Δy², respectively, generated by the single segment electrode shown in FIG. 4A;

FIGS. 7C and 7D are graphs of computer simulation results of the second spatial differences of the potentials, Δ²Vₑ/Δx² and Δ²Vₑ/Δy², respectively, generated by the electrode shown in FIG. 4C; and

FIGS. 8A and 8B are graphs of the spatial profiles of Δ²Vₑ/Δx² and Δ²Vₑ/Δy² along a specific line generated by each of the electrodes shown in FIGS. 4A-4E.

### DETAILED DESCRIPTION

The subject matter described herein includes electrode systems and related methods for providing therapeutic differential tissue stimulation. According to one aspect, an electrode system according to the subject matter described herein may include an electrode body and a plurality of electrode segments positioned along the electrode body. The electrode segments are predetermined shapes and sizes that produce a predetermined electrical field in an area surrounding the electrode body. Further, each of the electrode segments includes an outer surface which is exposed from the electrode body for coupling to tissue. A lead may be connected to each of the electrode segments for applying a common electrical signal to the electrode segments. Further an electrode array can consist of one or more electrodes, each electrode individually consisting of multiple segments. While the segments within an electrode are connected to a common electrical signal, the electrodes within an array may be connected to common electrical signals or differential electrical signals. The electrode(s) within an array may act as sources of current or voltage (anodes) or as sinks of current or voltage (cathodes). Further, the electrical signal generator may function as an electrode and may function as either an anode or cathode.

The predetermined electrical field can stimulate predetermined portions of tissue. An electrical signal generator may be connected to the electrode segments via the lead and configured to generate and deliver the common electrical signal to the electrode segments for therapeutic differential tissue stimulation.

In an exemplary application of the subject matter described herein, an electrode system according to the subject matter described herein may be used to electrically stimulate the nervous system for modulating neuronal activity. An electrode system may be applied to the nervous system for restoring function following neurological disease or injury. For example, deep brain stimulation may be used to treat movement disorders, including Parkinson's disease, by stimulation of brain nuclei. Further, the electrode systems according to the subject matter described herein may apply neuronal electrical stimulation for producing desirable clinical effects with reduced generation of unwanted side effects. The shapes and sizes of the electrode segments can be selected for producing an electrical field having a known shape and strength. Because the shape and strength of the electrical field can be known based on the shapes and sizes of the electrode segments, selective differential stimulation of different portions of tissue can be enabled. For example, an electrode system in accordance with the subject matter described herein can enable selective stimulation of different neuronal populations, based on their orientations, and thereby allow selective control of physiological function by electrical stimulation. Selective or differential stimulation can provide for the stimulation of neural elements to produce a desired clinical effect without stimulating those neural elements that produce unwanted side effect(s).

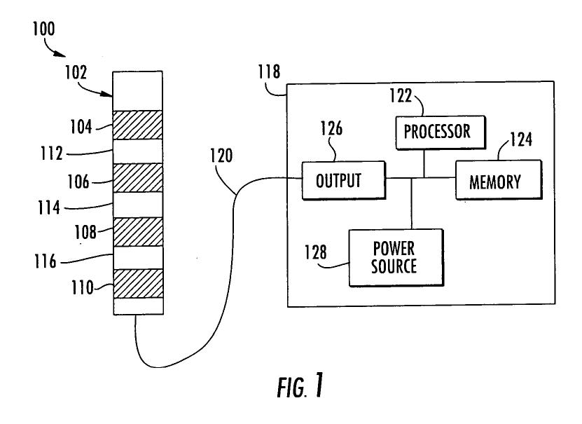

FIG. 1 illustrates a block diagram of an electrode system for providing therapeutic differential tissue stimulation according to an embodiment of the subject matter described herein. Referring to FIG. 1, an electrode system 100 may include an electrode body 102 and a plurality of electrode segments 104, 106, 108, and 110. Electrode segments 104, 106, 108, and 110 are electrically conductive and configured to generate an electric field in an area surrounding electrode body 102. Further, electrode body 102 may include a plurality of insulating sections 112, 114, and 116 positioned between electrode segments 104, 106, 108, and 110. The insulating sections may be made of non-conductive and/or semi-conductive materials. The separation of the electrode sections with insulating sections forms a segmented, conductive outer perimeter for each electrode segment. Adjacent pairs of electrode segments can be separated by a distance in the range of about 100 μm to about 1 cm. The coaxial length of electrode segments can be in a range of about 100 μm to about 1 cm.

In this embodiment, electrode segments 104, 106, 108, and 110 are ring-shaped. Alternatively, electrode segments may be any predetermined shape and size for generating a predetermined electrical field for stimulating predetermined portions of tissue as described in further detail herein. Exemplary shapes include a ring shape, a half ring shape, or some other substantial ring shape having a fraction of the circumference. Electrode segment may be made of any suitable conductive material. Further, any other suitable type, number, and combination of electrodes, insulating sections and other components may be used. Exemplary electrode segment material includes non-conductive material, semi-conductive material, stainless steel, platinum-iridium (Pt-Ir), iridium, oxides of iridium, titanium, nitride of titanium, conductive polymers (such as polyanalyine and polypyrole) combinations thereof and any other suitable conductive material. Further, the outer surface of electrode segments can have a substantially regular or irregular surface. Exemplary electrode segment shapes include a substantially ring shape, a substantially cylindrical shape, substantially spherical shape, and substantially hemispherical shape. Further, electrode segment may be recessed within insulating sections or a substrate by a predetermined recession depth.

An electrical signal generator 118 may be connected to electrode segments 104, 106, 108, and 110 and configured to apply a common electrical signal to the electrode segments for therapeutic differential stimulation of tissue. Generator 118 may be connected to electrode segments 104, 106, 108, and 110 via connector 120. Connector 120 may include a lead in its interior for electrically coupling to each electrode segment 104, 106, 108, and 110. Generator 118 may include a processor 122, a memory 124, an output module 126, and a power source 128. Further, generator 118 may include other components, other numbers of components, and other combinations of components suitable for applying an electrical signal to electrode segments. Generator 118 may include one or more output stages that regulate the current, voltage, or charge of each electrode. Each electrode within an array can be designated as a cathode, anode, ground, or floating, according to the programming of generator 118. Further, generator 118 can be programmed from outside the body via an electromagnetic signal, for example, radio frequency (RF), optical, or infrared. Memory 124 may be configured to store computer executable instructions and data for controlling the delivery of electrical pulses to electrode segments 104, 106, 108, and 110, although some or all of these instructions and data may be stored elsewhere. Processor 122 may receive instructions and data from memory 124 for controlling power source 128 and output 126 to generate and individually deliver electrical pulses to electrode segments 104, 106, 108, and 110. Output 126 is connected to electrode segments 104, 106, 108, and 110 via corresponding leads. Power source 128 is a battery, although other suitable types of power sources may be used. The application of electrical pulses to electrode segments 104, 106, 108, and 110 may be based on data about the positioning of portions of target tissue that are to be stimulated with an electrical field. The data about the positioning of the target tissue with respect to the electrode segments and dosage information about the target tissue can be stored in memory 124 and used for generating instructions for generating and applying a common electrical signal to the electrode segments such that a predetermined electrical field can be generated. The electrical field generated based on an applied electrical signal can be known such that particular portions of the tissue can be stimulated.

Electrode segments 104, 106, 108, and 110 represent a single electrode. In one embodiment, an electrode array can include additional segmented electrodes such that electrode body 102 has other segmented electrodes positioned along the electrode body. The electrode segments may receive separate electrical signals produced by generator 118. Each of the segmented electrodes may be connected to a corresponding lead for receiving a corresponding electrical signal from a plurality of output stages of generator 118. In this manner, multiple segmented electrodes can be positioned along an electrode body. Further, each of the segmented electrodes may receive an electrical signal from an electrical signal generator for producing a predetermined electrical field surrounding the electrode body.

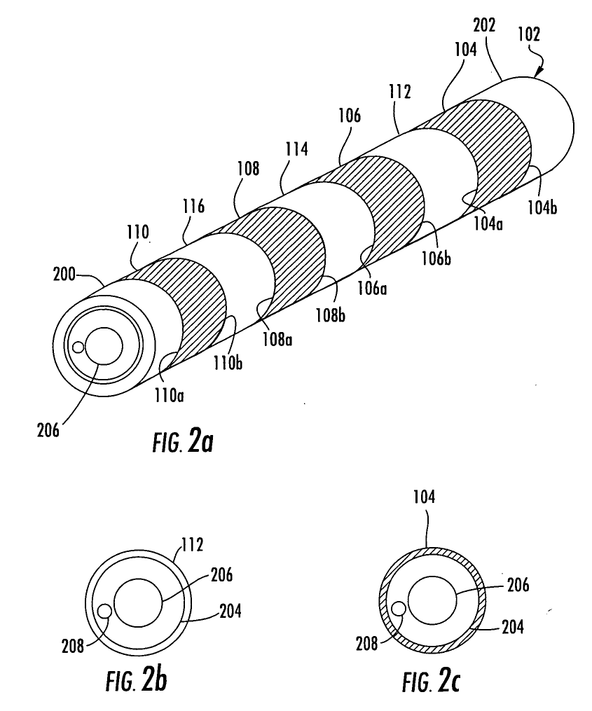

FIGS. 2A-2C illustrate more detailed views of electrode body 102 shown in FIG. 1. In particular, FIG. 2A illustrates a perspective view of electrode body 102. As set forth above, electrode segments 104, 106, 108, and 110 are electrically isolated from one another by insulating sections 112, 114, and 116. Electrode segments 104, 106, 108, and 110 include respective ends 104a/104b, 106a/106b, 108a/108b, and 110a/110b. In this example, the electrode segment ends are substantially planar, although the ends may have other shapes. Electrode body 102 may include ends 200 and 202 which are made of non-conductive material.

Referring to FIGS. 2B and 2C, cross-sectional views through insulating section 112 and electrode segment 104, respectively, of electrode body 102 are illustrated. A passage 204 extends through insulating sections 112, 114, and 116 and electrode segments 104, 106, 108, and 110 and through electrode body 102. A core 206 with a lead 208 is positioned in and extends along passage 204. Lead 208 is coupled to electrode segments 104, 106, 108, and 110. For example, FIG. 2C shows lead 208 coupled to electrode segment 104.

In another embodiment, electrode segments in accordance with the subject matter described herein may non-planar ends for increasing the perimeter-to-area ratios of the electrode segments. Electrode segments having increased perimeter-to-area ratios exhibit more non-uniform current densities on their surfaces. These electrode segments can be expected to have a higher activating function value than electrode segments with lower perimeter-to-area ratios. Such electrode segments can be more efficient than those with lower perimeter-to-area ratios by increasing the amplitude of the activating function generated per unit stimulus.

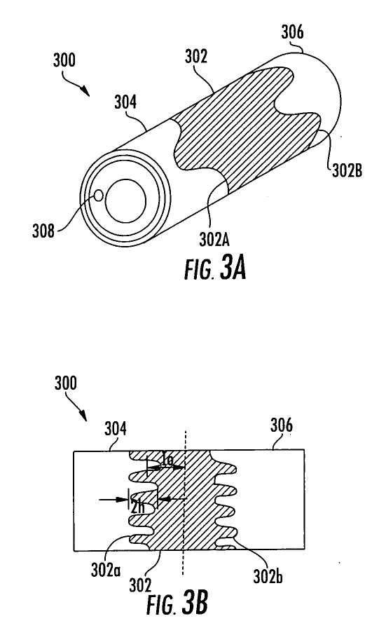

FIGS. 3A and 3B are perspective and top views, respectively, of a portion of an electrode body 300 including an electrode segment 302 having non-planar ends 302a and 302b according to an embodiment of the subject matter described herein. Referring to FIGS. 3A and 3B, ends 302a and 302b are serpentine in shape. Alternatively, the electrode segment ends may be any other non-planar shape. Electrode segment 302 is positioned between insulating sections 304 and 306. Further, electrode 302 may be one of a plurality of electrode segments having planar and/or non-planar ends in an electrode. Electrode 302 may be connected to lead 308, which may also be connected to one or more of the other electrode segments of the electrode system. The electrode segments may be separated by insulating sections. Increasing the amount of edge along the non-planar ends of the electrode segment increases the average current density.

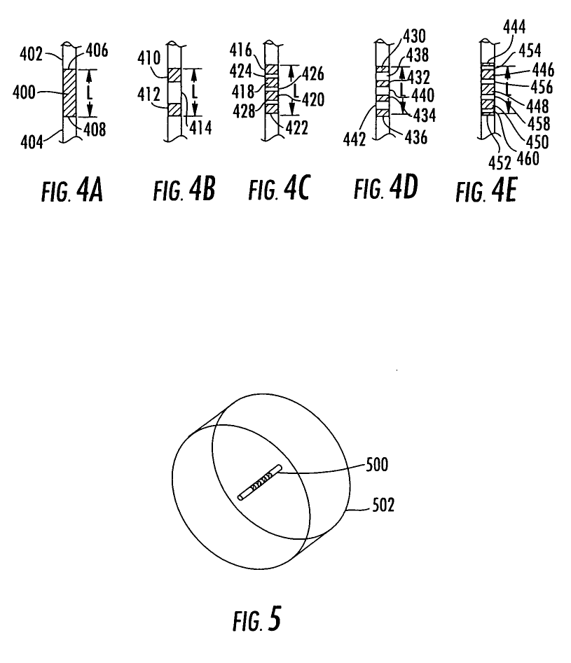

By selectively applying a common electrical signal to electrode segments of an electrode system in accordance with the subject matter described herein, the distribution of an electrical field around the electrode can be precisely distributed and controlled for stimulating predetermined portions of tissue. FIGS. 4A-4E are top plan views of portions of electrode bodies including one or more electrode segments having predetermined shapes and sizes according to the subject matter described herein. In particular, FIG. 4A illustrates a top view of a single electrode segment 400 and insulating sections 402 and 404. Electrode segment 400 has an axial length L from end 406 to end 408. Electrode segment 400 is ring-shaped, although the electrode segment may be any other suitable shape.

The electrode bodies shown in FIGS. 4B-4E include multiple electrode segments having different predetermined lengths and spacings. A common electrical signal may be applied to the electrode segments shown in FIGS. 4B-4E for generating distributed and controllable electrical fields. In particular, referring to FIG. 4B, electrode segments 410 and 412 and insulating section 414 have a combined axial length L, the same as electrode segment 400 shown in FIG. 4A. Electrode segments 410 and 412 collectively form a single electrode which is connected to a lead for receiving a common electrical signal to generate a distributed and controllable electrical field in an area surrounding the electrode segments. Electrode segments 410 and 412 are ring-shaped, although the electrode segments may be any other suitable shape.

Referring to FIG. 4C, electrode segments 416, 418, 420, and 422 are narrower than electrode segments 410 and 412 and spaced by insulating sections 424, 426, and 428. The combined lengths of electrode segments 416, 418, 420, and 422 and insulating sections 424, 426, and 428 are also equal to length L. Electrode segments 416, 418, 420, and 422 collectively form a single electrode which is connected to a lead for receiving a common electrical signal to generate a distributed and controllable electrical field in an area surrounding the electrode segments. Electrode segments 416, 418, 420, and 422 are ring-shaped, although the electrode segments may be any other suitable shape.

Referring to FIG. 4D, electrode segments 430, 432, 434, and 436 are narrower than electrode segments 416, 418, 420, and 422 and spaced by insulating sections 438, 440, and 442. The combined lengths of electrode segments 430, 432, 434, and 436 and insulating sections 438, 440, and 442 are also equal to length L. Electrode segments 430, 432, 434, and 436 collectively form a single electrode which is connected to a lead for receiving a common electrical signal to generate a distributed and controllable electrical field in an area surrounding the electrode segments. Electrode segments 430, 432, 434, and 436 are ring-shaped, although the electrode segments may be any other suitable shape.

Referring to FIG. 4E, electrode segments 444, 446, 448, 450, and 452 have different lengths. Further, the electrode segments are spaced by insulating sections 454, 456, 458, and 460. The combined lengths of electrode segments 444, 446, 448, 450, and 452 and insulating sections 454, 456, 458, and 460 are also equal to length L. Electrode segments 444, 446, 448, 450, and 452 collectively form a single electrode which is connected to a lead for receiving a common electrical signal to generate a distributed and controllable electrical field in an area surrounding the electrode segments. Electrode segments 444, 446, 448, 450, and 452 are ring-shaped, although the electrode segments may be any other suitable shape.

### Experimentation and Computer Simulations

Computer simulations were performed using the finite element method based on computer models of the electrodes shown in FIGS. 4A-4D. The tissue surrounding the electrode is modeled as a cylindrical object having a radius of 15 cm and a height of 15 cm. The outer boundary of the tissue model is set to 0 V. The electrode segments are set to 10 V, which is approximately three times larger than the average voltage used clinically, but the model was linear and both the current density and electric field intensity scale with the applied voltage. FIG. 5 illustrates a 3D numerical computer model 500 of the geometry of a 4-segment electrode system model having a total length of 7 cm between the end electrode segments and within a cylindrical object 502 for modeling tissue according to an embodiment of the subject matter described herein. The 3D model shown in FIG. 5 was partitioned into mesh elements by a finite element software package.

The nodal voltages (φ) were calculated by solving Laplace's equation:
∇²Φ=0.
Laplace's equation describes the potential variation in the electrolytic solution or tissue where the concentrations are uniform. The element current densities were derived from the nodal voltages with Ohm's law:
J=−σ·∇Φ.
The mesh size was set such that further refinement of the mesh resulted in less than 3% change in the total current delivered by the electrode. The total current delivered by the electrode was calculated by integration of the current density along the electrode surface.

The second spatial difference of the extracellular potential (the activating function, fαΔ²V) was used to estimate the effects of segmented electrodes having predetermined shapes and sizes on neuronal excitation. The activating function drives neuronal polarization by generating transmembrane currents in neurons, and has both positive components resulting in depolarization and negative components resulting in hyperpolarization. The activating function provides predictions on the activation patterns of neurons by extracellular sources. Distributions of Δ²Ve/Δx² (for neurons perpendicular to the electrode) and Δ²Ve/Δy² (for neurons parallel to the electrode) were calculated in the tissue medium from the nodal potentials of the finite element models where the mesh spacing (from ˜100 μm close to electrode to ˜2 cm far near the outer boundary) was used as the space step, Δx or Δy. This distribution of the activating function around the electrode was calculated from the finite element model for each of the segmented electrodes shown in FIGS. 4B-4E. The magnitude of the activating function varied across the electrodes. Increasing the number of electrode segments increased the magnitude of f. The electrode with the shortest length produced the largest magnitude of f.

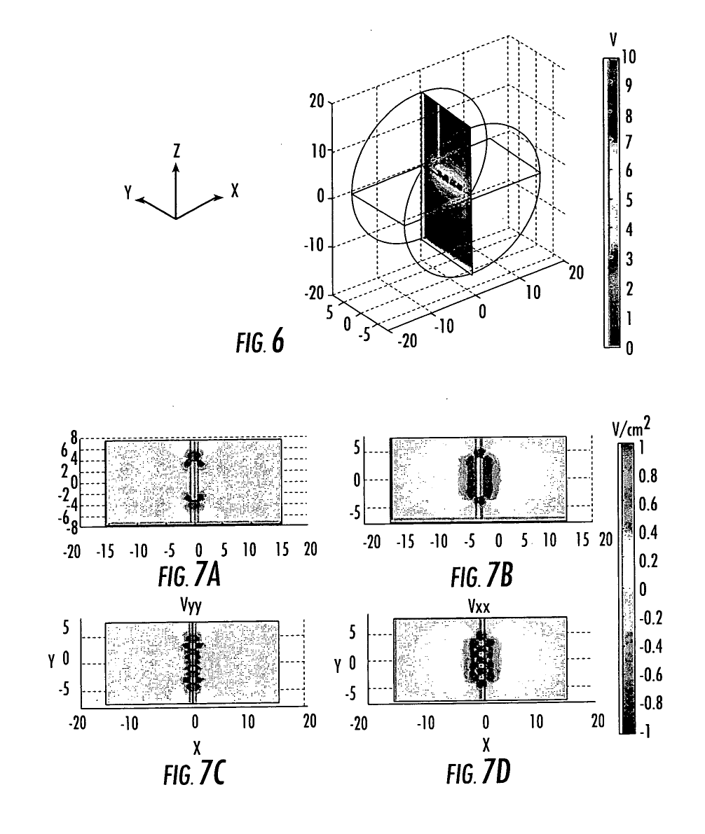

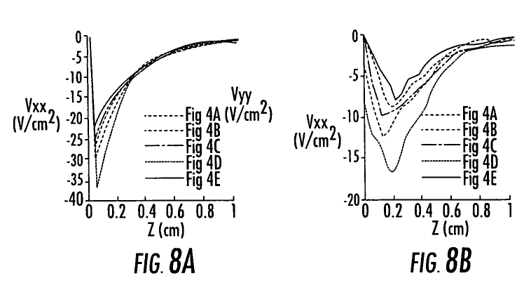

FIGS. 6-8B are computer simulation results based on computer models of the segmented electrodes shown in FIGS. 4A-4E. In particular, FIG. 6 is a 3D graph of voltage potentials generated in a surrounding tissue by a segmented electrode model based on the segmented electrode shown in FIG. 4C. The tissue surrounding the segmented electrode was modeled to have an electrical conductivity σ of 0.2 siemens per meter (S/m). As stated above, the simulation was performed with a finite element model with boundary conditions of 10 volts (V) on the electrode contacts and 0 V on the model boundary. FIGS. 7A-7D show the spatial distribution of the activating function for a single segment electrode (FIG. 4A) and a multiple segment electrode (FIG. 4C). FIGS. 8A and 8B show the magnitude and distribution of the activating function generated by the electrodes in FIGS. 4A-4E for neurons lying perpendicular and parallel to the log axis of the electrode, respectively.

FIGS. 7A-7D are graphs of computer simulation results illustrating the second spatial differences of the potentials, Δ²Vₑ/Δx² and Δ²Vₑ/Δy², generated by the electrode shown in FIG. 4A and the segmented electrode shown in FIG. 4C. In particular, FIGS. 7A and 7B illustrate the second spatial differences of the potentials, Δ²Vₑ/Δx² and Δ²Vₑ/Δy², respectively, generated by the single segment electrode shown in FIG. 4A. FIG. 7A shows Δ²Vₑ/Δy² in the axial (Y) direction (i.e., neurons parallel to the long axis of the electrode) in the Z=0 plane for the single segment electrode. FIG. 7B shows Δ²Vₑ/Δx² in the radial (X) direction (i.e., neurons perpendicular to the long axis of the electrode) in the Z=0 plane for the single segment electrode.

FIGS. 7C and 7D illustrate the second spatial differences of the potentials, Δ²Vₑ/Δx² and Δ²Vₑ/Δy², respectively, generated by the electrode shown in FIG. 4C. Δ²Vₑ/Δx² and Δ²Vₑ/Δy² have both positive components resulting in depolarization and negative components resulting in hyperpolarization of neurons surrounding the electrode. The spatial distributions of the activating function for neurons perpendicular to the electrode (Δ²Vₑ/Δx²) are similar for the electrodes shown in FIGS. 4A and 4C, while the activating function for neurons parallel to the electrode (Δ²Vₑ/Δy²) generated with segmented electrode has a larger spatial extent than with the electrode shown in FIG. 4A. FIG. 7C shows Δ²Vₑ/Δy² in the axial (Y) direction in the Z=0 plane for the 4-segmented electrode. FIG. 7D shows Δ²Vₑ/Δx² in the radial (X) direction in the Z=0 plane for the 4-segmented electrode.

FIGS. 8A and 8B are graphs illustrating the spatial profiles of Δ²Vₑ/Δx² and Δ²Vₑ/Δy² along a specific line generated by each of the electrodes shown in FIGS. 4A-4E. In particular, the graphs show the second spatial difference of the extracellular potentials (Δ²Vₑ/Δx²,Δ²Vₑ/Δy²) generated by each of the five segmented electrodes shown in FIGS. 4A-4E along a line radial to the electrode on the Y=3.25 cm plane (i.e., axial center of the most distal segment of the electrodes shown in FIGS. 4D and 4E). FIG. 8A shows Δ²Vₑ/Δx² (in the radial (X) direction, i.e., neurons perpendicular to the long axis of the electrode) as a function of the distance along Z from the electrode segments. FIG. 8B shows Δ²Vₑ/Δy² (in the axial (Y) direction, i.e., neurons perpendicular to the long axis of the electrode) as a function of the distance along Z from the electrode segments.

The computer simulation results demonstrate the difference in the spatial distribution of ƒ across the different segmented electrodes. Further, the computer simulation results demonstrate that the magnitude of the profiles generated by each of the segmented electrodes were larger than the profiles generated by the electrode having a single electrode segment shown in FIG. 4A. These results indicate that the magnitude of the stimulus required to produce a threshold level of membrane polarization is lower for segmented electrodes, and that the spatial patterns of stimulation can be controlled by applying a common electrical signal to segmented electrodes.

Electrode geometries that exhibit less uniform distributions of current density on their surfaces generate patterns of potential in tissue that exhibit greater spatial variation, and therefore generate larger values of activating function ƒ. Segmented electrodes (e.g., the segmented electrodes shown in FIGS. 4B-4E) generate larger magnitudes of activating function ƒ than the single segment electrode (e.g., the electrode shown in FIG. 4A). Further, segmented electrodes having short electrode segments (e.g., the segmented electrode shown in FIG. 4D) generate larger magnitudes of activating function ƒ than segmented electrodes having thicker electrode segments (e.g., the segmented electrode shown in FIG. 4C).

With the same stimulation intensity (electrode voltage), segmented electrodes (shown in FIGS. 4B-4E) generated larger magnitudes of Δ²Vₑ/Δx² and Δ²Vₑ/Δy² surrounding the conductive contact than the single segment electrode (shown in FIG. 4A), and thus required lower stimulation intensity than the single segment electrode to achieve the same level of neural activation. The electrode with 4 thin segments (shown in FIG. 4D) generated larger magnitudes of Δ²Vₑ/Δx² and Δ²Vₑ/Δy² than the electrode with 4 “thick”segments (shown in FIG. 4C). For different segmented electrode configurations with same segment length (shown in FIGS. 4D and 4E), the electrode with the larger insulative gap (shown in FIG. 4D) resulted in a larger magnitude of Δ²Vₑ/Δx² and Δ²Vₑ/Δy² surrounding the conductive contact than the electrode shown in FIG. 4E.

Segmented electrodes generated larger magnitudes of Δ²Vₑ in both the axial (e.g., shown in FIGS. 6 and 7A-7D) and radial directions (FIGS. 8A and 8B) implying that segmentation will increase functional stimulation coverage in both the axial and radial directions. The changes in the spatial distribution of the activating function will influence the selectivity of stimulation for differently oriented neural elements, for different types of neural elements (local cells vs. axons of passage), and for neurons lying at different distances from the electrode. Segmented electrodes generated patterns of electric field with greater spatial variation than did a large solid electrode, and more selective activation to targeted neural elements could be achieved by segmented electrodes with greater numbers of more densely spaced, smaller electrode segments. Further, several different stimulation channels may be achieved by using multiple segmented electrodes.

Other computer simulations were performed with segmented electrodes for characterizing current density distributions, field distributions, and impedances produced by the segmented electrodes. The computer simulation results demonstrate the effects of the number of segments, aspect ratio (length/radius) of each segment, total surface area and surface coverage (percentage of conductive surface area) of the electrode on the current density distributions, field distributions and electrode impedance.

It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. An electrode system for providing therapeutic differential tissue stimulation, the system comprising:
(a) an electrode body;
(b) a plurality of electrode segments positioned along the electrode body, wherein the electrode segments are formed from predetermined shapes and sizes that produce a predetermined electrical field for stimulating predetermined portions of tissue, and wherein each of the electrode segments includes an outer surface which is exposed from the electrode body for coupling to tissue; and
(c) a lead connected to each of the electrode segments for applying a common electrical signal to the electrode segments.

2. The system of claim 1 wherein the electrode body includes one or more insulating sections positioned between adjacent pairs of electrode segments.

3. The system of claim 2 wherein the one or more insulating sections comprise one of non-conductive and semi-conductive materials.

4. The system of claim 1 wherein an end of at least one of the electrode segments is substantially planar.

5. The system of claim 1 wherein an end of at least one of the electrode segments is substantially non-planar.

6. The system of claim 5 wherein the substantially non-planar end is substantially serpentine in shape.

7. The system of claim 1 wherein adjacent pairs of electrode segments are separated by a distance in the range of about 100 μm to about 1 cm.

8. The system of claim 1 wherein electrode segments have a length in arrange of about 100 μm to about 1 cm.

9. The system of claim 1 wherein electrode segments comprise a material selected from the group consisting of non-conductive material, semi-conductive material, stainless steel and platinum-iridium (Pt—Ir), iridium, oxides of iridium, titanium, nitride of titanium, and conductive polymers.

10. The system of claim 1 wherein electrode segments comprise a shape selected from the group consisting of a substantially ring shape, a portion of a substantial ring shape, a substantially cylindrical shape, substantially spherical shape, and substantially hemispherical shape.

11. The system of claim 1 wherein the electrical signal comprises at least one electrical pulse.

12. The system of claim 1 comprising an electrical signal generator connected to the lead and configured to apply the common electrical signal to the lead for delivering the common electrical signal to the electrode segments.

13. The system of claim 1 wherein the electrical signal generator is configured to apply the common electrical signal based on data regarding a position of tissue with respect to the electrode segments and tissue dosage information.

14. The system of claim 1 comprising:
(a) a plurality of sets of electrode segments positioned along the electrode body, wherein the electrode segments in each set are formed from predetermined shapes and sizes that produce a predetermined electrical field for stimulating predetermined portions of tissue, and wherein each of the electrode segments in each set includes an outer surface which is exposed from the electrode body for coupling to tissue; and
(b) a plurality of leads, wherein each lead is connected to electrode segments in a corresponding set for applying a common electrical signal to the electrode segments in the set.

15. A method for providing therapeutic differential tissue stimulation, the method comprising:
(a) coupling a plurality of electrode segments positioned along an electrode body to tissue, wherein the electrode segments are predetermined shapes and sizes that produce a predetermined electrical field for stimulating predetermined portions of tissue, and wherein each of the electrode segments includes an outer surface which is exposed from the electrode body for coupling to tissue; and
(b) applying a common electrical signal to the electrode segments.

16. The method of claim 15 wherein the electrode body includes one or more insulating sections positioned between adjacent pairs of electrode segments.

17. The method of claim 16 wherein the one or more insulating sections comprise one of non-conductive and semi-conductive materials.

18. The method of claim 15 wherein an end of at least one of the electrode segments is substantially planar.

19. The method of claim 15 wherein an end of at least one of the electrode segments is substantially non-planar.

20. The method of claim 19 wherein the substantially non-planar end is substantially serpentine in shape.

21. The method of claim 15 wherein adjacent pairs of electrode segments are separated by a distance in the range of about 100 μm to about 1 cm.

22. The method of claim 15 wherein electrode segments have a length in arrange of about 100 μm to about 1 cm.

23. The method of claim 15 wherein electrode segments comprise a material selected from the group consisting of non-conductive material, semi-conductive material, stainless steel and platinum-iridium (Pt—Ir), iridium, oxides of iridium, titanium, nitride of titanium, and conductive polymers.

24. The method of claim 15 wherein electrode segments comprise a shape selected from the group consisting of a substantially ring shape, a portion of a substantial ring shape, a substantially cylindrical shape, substantially spherical shape, and substantially hemispherical shape.

25. The method of claim 15 wherein applying a common electrical signal to the electrode segments includes applying at least one electrical pulse to the electrode segments.

26. The method of claim 15 using an electrical signal generator for applying the common electrical signal to the lead for delivering the common electrical signal to the electrode segments.

27. The method of claim 15 wherein applying a common electrical signal to the electrode segments includes applying the common electrical signal based on data regarding a position of tissue with respect to the electrode segments and tissue dosage information.

28. The method of claim 15 comprising:
(a) coupling a plurality of sets of electrode segments positioned along the electrode body to tissue, wherein the electrode segments in each set are formed from predetermined shapes and sizes that produce a predetermined electrical field for stimulating predetermined portions of tissue, and wherein each of the electrode segments in each set includes an outer surface which is exposed from the electrode body for coupling to tissue; and
(b) applying a plurality of common electrical signals to the electrode segment in the sets, wherein each common electrical signal is applied to a electrode segments in a corresponding set.
